(19) United States
(12) Patent Application Publication
(10) Pub. No.: US 2002/0099005 A1
(43) Pub. Date: Jul. 25, 2002

(54) Mucin comprising vehicle for the transport of biologically-active agents

(76) Inventors: Ashok Kumar Shukla, Wood Stock, MD (US); Mukta Shukla, Wood Stock, MD (US); Amita Shukla, Wood Stock, MD (US)

Correspondence Address: Ashok K. Shukla, 10316 Kingsway Court, Woodstock, MD, 21163, US

(21) Appl. No.: 09/767,462

(22) Filed: Jan. 23, 2001

(51) Int. Cl. A61K 48/00; A61K 38/16

(52) U.S. Cl. 514/8; 514/44

(57) ABSTRACT

In the present invention we describe a vehicle for the transport of biologically active or therapeutic agents into organisms, such as human beings. The vehicle described in the present invention comprises mucin, wherein said mucin is present in a natural or modified form. The mucin component of the vehicle, as described in the present invention, serves to enhance the transport of biologically active agents, such as therapeutic agents into living organisms; to control and/or improve the delivery of biologically active agents to cells, tissues, organs or organelles; to increase the level of specificity in targeting particular cells or cells types; and/or, to enhance the activity of such therapeutic agents once they enter an organism. The vehicle described in the present invention is used to carry and deliver biologically active agents and can be used for biochemical, biomedical, therapeutic, clinical, or other applications in organisms and cells including, but not limited to, delivery of DNA, RNA, PNA, polynucleotides and proteins into cells, tissues or organisms; gene delivery applications; in vivo gene therapy, ex vivo gene therapy or in vitro gene therapy; customized therapeutics; vaccination of organisms; genetic vaccination of organisms; and, delivery of pharmaceutical products or biologically active chemical, biochemical or biological agents into cells and organisms.

### FIELD OF THE INVENTION

In the present invention we describe a vehicle for the transport of biologically active or therapeutic agents into organisms, such as human beings. The vehicle described in the present invention comprises mucin, wherein said mucin is present in a natural or modified form. The mucin component of the vehicle, as described in the present invention, serves to enhance the transport of biologically active agents, such as therapeutic agents into living organisms; to control and/or improve the delivery of biologically active agents to cells, tissues, organs or organelles; to increase the level of specificity in targeting particular cells or cells types; and/or, to enhance the activity of such therapeutic agents once they enter an organism. The vehicle described in the present invention can also contain any other components, such as lipids, proteins or other molecules used as a means of performing the transport and delivery of desired biologically active agents. The vehicle described in the present invention is used to carry and deliver biologically active agents such as chemicals, small molecules, pharmaceutical drugs, biomolecules, proteins, peptides, DNA (deoxyribonucleic acid), RNA (ribonucleic acid), PNA (peptide nucleic acid), polynucleotides, proteins, peptides, lipids, glycoproteins, glycolipids, carbohydrates or a combination thereof into living organisms.

The mucin-based delivery vehicle described in the present invention can be used for biochemical, biomedical, therapeutic, clinical, or other applications in organisms and cells including, but not limited to, delivery of DNA, RNA, PNA, polynucleotides and proteins into cells, tissues or organisms; gene delivery applications; in vivo gene therapy, ex vivo gene therapy or in vitro gene therapy; customized therapeutics; vaccination of organisms; genetic vaccination of organisms; and, delivery of pharmaceutical products or biologically active chemical, biochemical or biological agents into cells and organisms. Since current delivery methods and tools, especially those used in drug delivery, vaccination and gene therapy, present a number of limitations and disadvantages, the present invention offers tremendous potential for providing an effective, new method for highly targeted delivery of biologically active agents.

### BACKGROUND OF THE INVENTION

The fields of biomedicine and therapeutics rely on the effective delivery of therapeutic agents into organisms. Therapeutic agents, as described in the present invention include biologically active chemical, biochemical or biological agents derived from a natural source, agents derived from a natural source and modified, or agents produced synthetically. Traditional therapeutic agents include pharmaceutical drugs and therapeutics, extracted from nature or developed synthetically, which are used to treat a wide spectrum of diseases and medical conditions in living organisms. Additionally such therapeutic agents can be introduced into an organism for a preventative application such as vaccination against the manifestation of a disease or medical condition.

In addition to chemical and chemical-based pharmaceutical products, more recently, therapeutic agents have included biomolecules such as proteins, peptides, hormones, enzymes, antibodies and other biological molecules for the treatment of different diseases and for the vaccination of an organism against disease. Most recently, the field of therapeutic agents has also expanded to include nucleic acids such as DNA and RNA as part of the newly emerging field of gene therapy, where genetic material is inserted into cells, tissues or an organism for a short-term, or ideally, more long-term therapeutic effect. Biologically active agents, as described in the present invention, include any elements, compounds or biological substances, such as biomolecules, where said agents are involved, in any capacity, in biological functions, processes or activities in cells, tissues or organisms.

Drug Delivery

While a number of delivery methods currently exist for the delivery of therapeutic agents into living beings, most still present significant limitations. Additionally, more sophisticated methods are needed for the delivery of larger, more complex molecules and biomolecules such as proteins. An ideal drug delivery technology would provide for targeted, sustained release with minimally invasive or non-invasive delivery methods and maximum ease of use. Currently available delivery methods still face significant limitations and do not provide all of the features of an optimal delivery vehicle for therapeutic agents.

At present, biologically active agents such as pharmaceutical drugs and biomolecules are delivered through a variety of methods including orally, nasally, through the lungs, through the eyes, through the skin and intravenously. The active ingredient of a pharmaceutical product is delivered in its pure form or in complex with delivery molecules and components that optionally enhance the transport, delivery and absorption of the active ingredient in a living being.

Pharmaceutical products that are delivered orally, in solid or liquid form, must pass through the digestive system, where the therapeutic is absorbed by the organism. While this method is effective for the delivery of many chemical agents, biological therapeutic agents such a proteins and enzymes can be easily degraded during the digestive process such that they are denatured by the digestive process prior to exerting their therapeutic effects.

Furthermore, for many therapeutic agents for the effective treatment of diseases it is necessary that an organism obtain a relatively constant dose of a biologically active agent over an extended period of time. This requires that an organism obtain multiple doses of that biologically active agent during the course of treatment. While some methods have been developed in which synthetic polymers are conjugated with biologically active agents to slow down their rate of release in an organism, such methods are limited in the extent to which they can provide constant dosage and present toxicity threats due to the presence of synthetic polymers and other delivery components complexed with the biologically active agents.

In addition to oral delivery, many pharmaceutical products are introduced into organisms through inhalation, such that the therapeutic agents enter an organism through its respiratory system. Yet, only a limited number of therapeutic agents can be delivered into an organism using this method since many larger molecules are not suitable for inhalation and cannot be absorbed by the respiratory system. In addition, to the best of our knowledge, no suitable vehicles exist in the market for effective delivery of larger therapeutic agents such as biomolecules.

Another method for therapeutic agents delivery is intravenously. This method can be used to deliver many different types of therapeutic agents and constant intravenous delivery through an intravenous apparatus or device can ensure constant delivery of a agent over an extended period of time. Yet, one of the main disadvantages of this method is that it requires an invasive procedure and in the case of human therapeutics, in particular, often requires that a medical professional administer the therapeutic to a patient requiring treatment.

Recently, transdermal technologies have also been tested for the delivery of therapeutic agents and biomolecules into living organisms. Yet, currently no ideal technologies exist to control the rate of delivery and the dosage of a therapeutic agent that is delivered to an organism, thus limiting its utility.

For therapeutic agents, an ideal delivery method controls the rate of delivery such that a living being receives a regulated dose of the drug over an extended period of time. Such controlled delivery or slow release methods enhance the therapeutic efficacy of many therapeutic products. Additionally, an ideal delivery method is minimally invasive or non-invasive. Furthermore, current drug delivery methods are significantly limited by the extent to which specific therapeutic agents can be targeted to enter specific types of cells, tissues, organs or organelles.

An additional factor that is important for optimal therapeutic agents delivery is having delivery vehicles that present minimal or no side effects due to the vehicle used to deliver therapeutic agents to an organism. For example, when an therapeutic agent is administered to an organism using an additional delivery vehicle used to carry or transport the therapeutic agent, effective delivery depends on the vehicle presenting no toxicity to the organism. For example, liposomes, or cationic lipids, which are currently used to deliver many therapeutic agents, are often synthetically synthesized and present toxicity risks to organisms and their cells or tissues.

Thus, the field of drug delivery could benefit significantly from a biologically active agents delivery vehicle that is effective, specific and non-toxic in delivering specific biologically active agents to organisms, such as human beings.

Gene Therapy

In recent years, gene therapy has become an increasingly important component of therapeutics, especially in organisms such as humans. Gene therapy is the treatment of diseases and disorders in organisms through the replacement, repair or alteration of defective genes and/or their defective gene products, such as peptides and proteins. Thus, in gene therapy, genes, which are composed of DNA, need to be delivered to cells so that they can subsequently be expressed in cells such that they result in the production of desired proteins and enzymes or in the replacement, restoration or repair of defective genes. The entire fields of gene therapy and gene delivery depend on the availability of a universal gene delivery vector or mechanism that effectively delivers polynucleotides such as DNA, RNA and PNA into cells with high specificity and, ideally, no toxicity.

Gene therapy can be performed in vivo (genes are directly inserted into a host organism for delivery to that organism's cells), in vitro (cells are removed from an organism and gene delivery into those cells is performed outside the organism) and ex vivo (cells are removed from an organism, genes are inserted into those cells outside the organism and these cells are then re-introduced into the organism). Current gene delivery methods include calcium phosphate precipitation, the use of cationic lipid-DNA complexes, liposomes, electroporation and the use of viral vectors. Yet, each of these methods offers distinct disadvantages.

Calcium phosphate precipitation does not always result in sufficient levels of gene delivery into cells and offers very low specificity. Cationic lipids, or positively charged lipids, which are combined in a complex with DNA, are often toxic to cells and thus ineffective for in vivo gene therapy. Liposomes, lipid bi-layer vesicles carrying DNA, which are inserted into cells, also potentially present toxicity, in addition to providing low target cell specificity and insufficient levels of DNA delivery in many instances. Electroporation is a method where very high voltage levels are used to transport genes into cells. Since DNA is highly negatively charged, the application of such an electric current allows for the passage of DNA into cells. Yet, this method cannot be used in in vivo gene delivery and at high voltage levels the death rate of cells is significantly high, limiting the scope of this method.

In viral vector transfection, a virus infects cells, delivering the DNA it contains to those cells. Since viruses can be modified and altered to contain foreign genes or genes needed to perform gene therapy, they can be used to transport DNA into specific cells. In fact, under ideal circumstances, viral vectors can be modified such that the virus retains its gene delivery mechanism but the pathogenic components of its genome are removed. Although viral vectors can be used in vivo, one of their main disadvantages is that they can transform in an organism causing potential harmful effects such as an infection or a strong immunogenic response. The potential of creating an immunogenic response has severely limited the efficacy of viral vectors for gene therapy.

Vaccines

The process of vaccination has been used for the prevention and eradication of many diseases including smallpox, polio, typhus, tetanus and hepatitis A and B. In vaccination a vaccine is administered to an organism to prevent, ameliorate or treat a specific disease. A vaccine typically consists of a preparation of attenuated, weakened or killed pathogens, such as bacteria or viruses, or parts of the structure or body of said pathogens. Many currently available vaccines depend on the delivery of the immunity conferring agents, often pathogen protein and peptide fragments, to an organism. Upon administration the pathogen components of the administered vaccine, known as antigens, stimulate a protective immune response in the host organism that serves to protect the host organism from future, more adverse infections by the same pathogen.

An immune response in an organism is typically mediated by many different cellular and cytolytic parts of the host organism's immune system. The two main arms of the immune system are the humoral arm and the cytolytic arm. The humoral arm is mediated by B lymphoid cells, which produce antibodies and release them into the interstitial fluids where they bind to foreign pathogen proteins, thus eradicating them or marking them for destruction by other cells. T lymphoid cells, also known as cytotoxic T cells, mediate the cytolytic arm. When a pathogen or its components enter the host organism's cells, those cells display fragments of the pathogen's proteins on their surface. T cells recognize foreign protein displayed on the organism's cells and act to destroy those cells, thus eliminating the pathogen that has invaded host cells. In addition to these two main arms of the immune system, many other cellular components are involved in generating an immune response.

During vaccination, the immune system of a host organism's body generates an immune response to the introduced pathogen or pathogen components, thus priming the body's immune system. Ideally, the quantity and type of pathogen or pathogen particles introduced into an organism is sufficient to generate an immune response that confers immunity from subsequent infections but insufficient to generate significant symptoms or manifestations of the disease caused by that pathogen. One of the areas of potential improvement for vaccines relies on the development of more effective methods for delivering attenuated pathogens, and pathogen proteins, peptides and other pathogen biomolecules into organisms.

Genetic Vaccines

While currently available vaccination methods and technologies are well suited for generating immunity against many known pathogens, they are not well suited for many other applications due to the limitations of the immune response generated by an organism in response to exposure to only fragments of the pathogen's structure or attenuated pathogens. Often, only either the humoral or cytolytic arm of the immune system is activated and long-term or even effective immunity is not conferred. In fact, many diseases including AIDS, malaria, herpes and cancer could potentially be prevented or treated through the use of vaccines, albeit not through existing vaccine technologies.

In order to develop vaccines and treatments for many of the aforementioned diseases, many new vaccine technologies are currently being explored. One of the latest technologies is genetic vaccines. Genetic vaccines are basically gene-based vaccines in which genes coding for specific proteins or structural components of a pathogen are introduced into the cells of a host organism. Once the newly introduced genes enter the host cells, the expression of those genes leads to the production of proteins and other components of the pathogen. Subsequently, the host's immune system responds to the pathogen proteins, generating a protective immune response.

One of the most attractive features of genetic vaccines is that they can be designed such that only specific, desired proteins from the pathogen enter the host. These proteins would be those specific components of the pathogen that the host recognizes as an antigen and targets for destruction by the immune system. Not only does this method ensure that desired pathogen proteins enter the host but it also prevents the introduction of other, potentially harmful components of the pathogen. Furthermore, genetic vaccines can be used effectively to activate both the humoral and cytolytic arms of the immune system. Yet, as with gene therapy, one of the greatest limiting factors with genetic vaccines is the availability of a suitable gene delivery vehicle. Currently many of the same methods are used for genetic vaccines as for gene therapy and, as discussed, each one of these methods offers significant limitations.

Mucin for the Delivery of Therapeutic or Biologically active Agents

Since current delivery methods and tools are limited in their scope, efficacy and utility, there is a strong need for a nontoxic, safe, effective and vehicle for the targeted delivery of many different types of therapeutic agents into living organisms. The delivery vehicle described in the present invention solves many of the problems associated with current biologically active agents transport and delivery methods and it thus provides a valuable tool for delivering biologically active agents to cells and organisms for different applications.

Also, since the specificity of currently available delivery methods for therapeutic agents is very limited, there is a strong need for a delivery vehicle that can provide very high specificity in identifying specific cells as the targets of delivery. The mucin-containing delivery vehicle for biologically active agents, as described in the present invention, also provides very high specificity and thus uniquely combines many of the advantageous features of an optimal biologically active agents delivery vehicle.

Mucins are glycoproteins with a very high molecular weight, up to several tens of millions of Daltons. Glycoproteins are proteins with carbohydrate molecules attached to them. Mucins are typically from 50 to 90 percent carbohydrate by composition and they are generally water-soluble. In mucin, the carbohydrate molecules are attached as chains to the backbone of the proteins. Since carbohydrates are generally linear molecules the resulting structure can be likened to that of a baby bottle brush, with the carbohydrate molecules forming individual prongs radiating from the central protein backbone.

Mucins are typically found in the mucus of organisms of many different species. For example, in most mammalian species mucins are secreted from epithelial cells of the gastrointestinal, respiratory and genitourinary tracts to form mucus gels that line organs such as the mouth, esophagus, stomach, intestines, lungs and other organs. Mucins are rapidly secreted by goblet, glandular and other cells types and they serve a number of different functions. For example, in the lungs, mucins bind bacteria and other microorganisms, facilitating their mucociliary clearance. Alternatively, mucins can also be present in a membrane-bound form on the surface of different types of cells.

In organisms, mucins are found in monomeric, dimeric and polymeric forms. In the latter forms nucin monomers are held together by bonds, such as disulfide bonds. Mucins are subject to extensive post-translation glycosylation and polymerization resulting in large complexes, or gels, of many monomeric mucin units.

The physical, biological, chemical and biochemical properties of mucin are well suited for its role as a transport and delivery vehicle for biologically active agents. The many different types of carbohydrate chains present on the protein backbone of a mucin unit provide the potential for high levels of specificity when targeting specific cell types. Furthermore, the physical and chemical structures of mucin molecules are well suited for entangling and subsequently transporting and delivering biologically active agents. Such biologically active agents can be bound to mucin through physical entanglement, electrostatic forces, or different types of bonds such as covalent bonds.

In addition, the mucin component of the biologically active agents delivery vehicle described in the present invention can be treated with enzymes such as endoglycosidases or exoglycosidases to enhance the binding and delivery capabilities of mucin. Furthermore, the presence of a plurality of different types of carbohydrates on the side chains of mucins offers the potential for creating different types of customized mucins, each suited for a specific delivery application using a specific biologically active agent.

Mucin, as used in the delivery vehicle described in the present invention, can be derived from a natural source or produced synthetically. Naturally or biologically-derived mucin offers strong advantages since it minimizes risks of toxicity and an unfavorable immunogenic response in the organism receiving a biologically active agent. Furthermore, mucins are well suited for specific therapeutic methods such as controlled or extended delivery of certain biologically active agents since the transport, delivery and degradation of mucins in an organism is well managed. Thus, mucins present a very attractive tool for the delivery of biologically active agents.

Advantages of the Invention

Mucin's physical, chemical and biological properties make it a very favorable vehicle or vehicle component for the transport and delivery of biologically active agents. In the present invention we describe a delivery vehicle for the transport of biologically active agents where said delivery vehicle is comprised of the desired biologically active agents; one or more different types of mucin; and, optionally, additional molecules that may enhance the transport and delivery functions of the vehicle. Mucins are particularly advantageous for the transport and delivery of biologically active agents delivery because they:

can maintain the stability of biologically active agents during the transportation and delivery process;

are capable of binding to, carrying and delivering to cells, small biologically active agents, as well as, large, complex therapeutic agents, such as nucleic acids;

can be very specifically modified for recognition by specific cells;

can provide targeted delivery to specific cell types;

do not present the potential for generating an immune response as viral vectors for gene therapy do;

pose low or no toxicity risks to the organism to which the biologically active agents are being administered;

can be derived directly from an organism and used for biologically active agents delivery in the same or a different organism;

are degraded once they enter desired cells, thus making them suitable carrier vehicles;

are well-suited for in vivo, in vitro and ex vitro delivery of biologically active agents;

can be utilized for slow, sustained or controlled delivery of biologically active agents;

can be produced large-scale for many different biomedical, biologically active and other applications.

The various features of novelty, which characterize the present invention, are pointed out with particularity in the claims annexed to and forming a part of this disclosure. For a better understanding of the invention, its advantages and objects, reference is made to the accompanying drawings and descriptive matter in which a preferred embodiment of the invention is illustrated.

### BRIEF DESCRIPTION OF THE DRAWINGS

The foregoing and still other objects of this invention will become apparent, along with various advantages and features of novelty residing in the present embodiments, from study of the following drawings, in which:

FIG. 1 is an expanded view of one embodiment of a biologically active agents transport vehicle comprising mucin and a biologically active agent, according to the present invention.

FIG. 2 is an expanded view of one embodiment of a biologically active agents transport vehicle comprising mucin and a biologically active agent physically entangled in mucin, according to the present invention.

FIG. 3 is an expanded view of one embodiment of a biologically active agents transport vehicle comprising mucin and a biologically active agent covalently bound to mucin, according to the present invention.

FIG. 4 is an expanded view of one embodiment of a biologically active agents transport vehicle comprising mucin; a biologically active agent; and, additional molecules, according to the present invention.

FIG. 5 is an expanded view of one embodiment of a biologically active agents transport vehicle comprising mucin; a biologically active agent; and, additional molecules serving as conjugates between mucin and the biologically active agent, according to the present invention.

### DESCRIPTION OF THE PREFERRED EMBODIMENTS

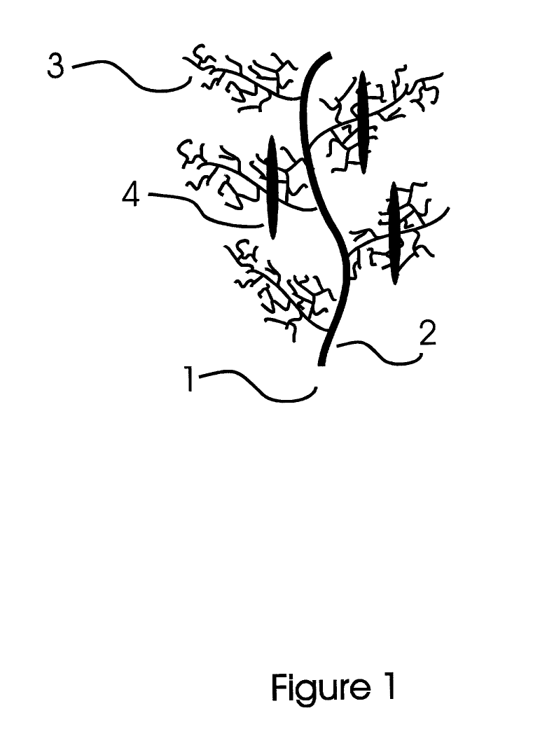

FIG. 1 shows a biologically active agents transport vehicle comprising mucin and a biologically active agent, according to the present invention. As is shown in FIG. 1, mucin (1) consists of a protein backbone (2) with side chains (3), comprised of carbohydrates, attached to the backbone (2). As is also shown in FIG. 1, to form the delivery vehicle for biologically active agents, the biologically active agents (4) are in contact with the protein backbone (2) and or carbohydrate side chains (3) of the mucin component (1) of the delivery vehicle.

While FIG. 1 shows a mucin monomer in contact with subunits of a biologically active agent, any component or configuration of mucin can be used for the transport vehicle described in the present invention including, but not limited to, a mucin type selected from the group comprised of mucin monomers, mucin dimers of the same type of mucin, mucin trimers of the same type of mucin, mucin polymers of the same type of mucin, mucin diners of different types of mucin, mucin trimers of different types of mucin, mucin polymers of different types of mucin, mucin copolymers, mucin homopolymers, linear mucin polymers, branched mucin polymers, cross-linked mucin polymers, mucin components, mucin subcomponents, mucin particles, mucin fragments, mucin protein backbone, mucin carbohydrate side chains, mucin conjugates, mucin conjugates with lipids, mucin conjugates with proteins, mucin conjugates with peptides, mucin conjugates with carbohydrates, and combinations thereof. Different types of mucin, as described above, are mucins that differ from each other in their source; in their chemical, physical, biological or biochemical composition or properties; or in the means by which, or the extent to which the mucins have been modified.

Mucin (1), as described in the present invention, can be derived from any natural, biological source. For example, mucin can be obtained from human mucus secreted in the gastrointestinal system, respiratory system or genitourinary system. Mucin can also be obtained from any eukaryotic or prokaryotic organisms that produce or secrete mucin. Mucin obtained from such biological sources, or native mucin, can be modified, transformed, purified, altered or used in its natural state for the means of transporting biologically active agents, according to the present invention. Furthermore, mucin can be derived from any non-biological sources, non-living organisms, or produced synthetically. Thus, mucin (1) can be comprised of one or more different types of mucin including, but not limited to, mucin selected from the group comprised of native mucin; mucin from a biological source; mucin from a natural source; mucin from a living organism; mucin from a non-living organism; synthetically produced mucin; modified mucin; physically altered mucin; chemically altered mucin; biochemically altered mucin; biologically altered mucin; enzyme modified mucin; electrochemically altered mucin; purified mucin; mucin obtained from humans; mucin obtained from plants; mucin obtained from living organisms; mucin purified from humans; mucin purified from plants; mucin purified from living organisms; and, combinations thereof.

The mucin (1) obtained from any source, natural or synthetic, can be any modified mucin including, but not limited to, modified mucin selected from the group comprised of biologically modified mucin; physically modified mucin; chemically modified mucin; enzyme modified mucin; mucin modified by variations in temperature; heat modified mucin; cold modified mucin; mucin modified by the alteration of electric charges on mucin; mucin modified by the alteration of electric charges on mucin protein components; mucin modified by the alteration of electric charges on mucin carbohydrate components; mucin modified by changes in pH; mucin modified by the addition of carbohydrate molecules to said mucin; mucin modified by the removal of carbohydrate molecules from said mucin; mucin modified by the addition of protein molecules to said mucin; mucin modified by the removal of protein molecules from said mucin; mucin modified by the addition of biomolecules to said mucin; mucin modified by the addition of lipid to said mucin; mucin modified by the application of pressure to said mucin; mucin modified by the application of an electric current to said mucin; and, combinations thereof.

The mucin (1) can be modified prior to its combination with biomolecules, during its combination with biomolecules or after its combination with biomolecules. Furthermore, as described above, the modifications performed on mucin can be any modifications including the removal, alteration or addition of carbohydrate or protein components to the protein backbone (2) and carbohydrate side chains (3) of mucin (1). For example, mucin (1) can be modified by the addition or removal of different monosaccaride groups from its carbohydrate side chains or by the addition or removal of carbohydrates such as sialic acid. The latter modification can be performed by enzymes, such as sialidases.

Mucin can be modified such that its specificity is enhanced. Specificity enhancements include higher specificity in binding, transporting and delivering specific biologically active agents; specificity in delivering desired therapeutic agent to specific, targeted cells, tissues, organelles, organs or organisms; and, specificity in the physical, chemical, biological and biochemical properties of mucin. For example, the treatment of the mucin (1) with enzymes can alter certain mucin molecules so that they become more effective in binding and/or transporting specific therapeutic agents.

Furthermore, specific carbohydrates can be added to mucin such that the added carbohydrates are recognized by specific cell types in an organism, resulting in targeted delivery of a biologically active agent by the mucin based vehicle, described in the present invention. Specifically, mucin can be modified such that the delivery vehicle, described in the present invention, is recognized by receptors on specific cell types or tissues. When the vehicle's receptor specific moieties are recognized by specific receptors in desired target cells, the delivery vehicle can enter those cell types or tissues through endocytosis. Endocytosis is the process whereby a cell adheres a certain molecule or complex to its exterior cell membrane and then engulfs it to introduce that molecule or complex into the interior of the cell. For example, when sialic acid is removed from mucin, galactose molecules become the terminal molecules of the mucin carbohydrate chains. Galactose is better recognized by cell surface galactose receptors, thus resulting in more effective endocytosis of the delivery vehicle.

The biologically active agent shown in FIG. 1 is comprised of at least one type of any biologically active agent including, but not limited to, those selected from the group comprised of biologically active chemical compounds, deoxyribonucleic acid, ribonucleic acid, peptide nucleic acid, polynucleotides, nucleic acids, genes, antisense deoxyribonucleic acid, antisense ribonucleic acid, ribozymes, proteins, peptides, amino acids, lectins, enzymes, lipids, carbohydrates, polysaccharides, mucopolysaccharides, lipoproteins, glycoproteins, glycolipids, inhibitors, biological response modifiers, hormones, antibodies, monoclonal antibodies, antigens, biologically active chemical compounds, vitamins, cells, cell fragments, biotechnology drugs, blood products, cardiovascular agents, central nervous system agents, gastrointestinal agents, respiratory agents, aceinhibitors, alkaloids, antacids, analgesics, anabolic agents, anti-cancer agents, anti-infective agents, anti-fungal agents, anti-viral agents, anti-bacterial agents, anti-protozoan agents, anti-parasitic agents, anti-tubercular agents, anti-inflammatory agents, antineoplastic agents, anti-allergic agents, histamines, anti-histamines, anti-coagulation agents, circulatory drugs, metabolic potentiators, anti-anginal agents, anti-diabetic agents, anti-rheumatic agents, narcotics, opiates, cardiac glycosides, neuromuscular blockers, sedatives, local anesthetics, general anesthetics, radioactive compounds, prodrugs, anti-arthritic agents, analgesic agents, anti-emetic agents, dermatological agents, immunosuppressive agents, ophthalmic agents, ocular agents, anti-arrhythmia agents, anti-asthmatic agents, anti-cholesterolemics, anticonvulsants, anticoagulants, antidepressants, anti-diarrheal preparations, anti-emetics, anti-hypertensives, antilipid agents, anti-manic agents, anti-migraine agents, anti-nauseants, anti-psychotic agents, anti-stroke agents, anti-thyroid preparations, anabolic drugs, anti-obesity agents, antipyretic agents, antispasmodic agents, anti-thrombotic agents, anti-tumor agents, anti-ulcer agents, anti-uremic agents, anxiolytic agents, appetite stimulants, appetite suppressants, beta-blocking agents, bronchodilators, cardiovascular agents, cerebral dilators, chelating agents, chemotherapeutic agents, cognition activators, contraceptives, coronary dilators, cough suppressants, decongestants, deodorants, dermatological agents, diabetes agents, diuretics, emollients, erythropoietic drugs, expectorants, fertility agents, growth regulators, hormone replacement agents, hyperglycemic agents, hypoglycemic agents, ion-exchange resins, laxatives, migraine treatments, mineral supplements, mucolytics, narcotics, neuroleptics, neuromuscular agents, non-steroidal anti-inflammatory agents, nutritional additives, peripheral vasodilators, prostaglandins, psychotropic agents, renin inhibitors, respiratory stimulants, sedatives, steroids, stimulants, sympatholytics, thyroid preparations, tranquilizers, uterine relaxants, vaginal preparations, vasoconstrictors, vasodilators, anti-vertigo agents, wound healing agents, and combinations thereof.

Biologically active chemical compounds, also biomolecules, are defined as compounds that are obtained from biological sources or synthesized artificially and which play an essential role in a physical, chemical or biochemical interaction or reaction. Furthermore, the biologically active agent (4) can consist of one or more different types of biologically active agents including, but not limited to, those selected from the group comprised of native biologically active agents, biological source derived biologically active agents, synthetically produced biologically active agents, modified biologically active agents, physically altered biologically active agents, chemically altered biologically active agents, electrochemically altered biologically active agents, enzyme modified biologically active agents, purified biologically active agents, and therapeutic agents. Basically, the biologically active agents can be from a natural state, modified, or produced synthetically.

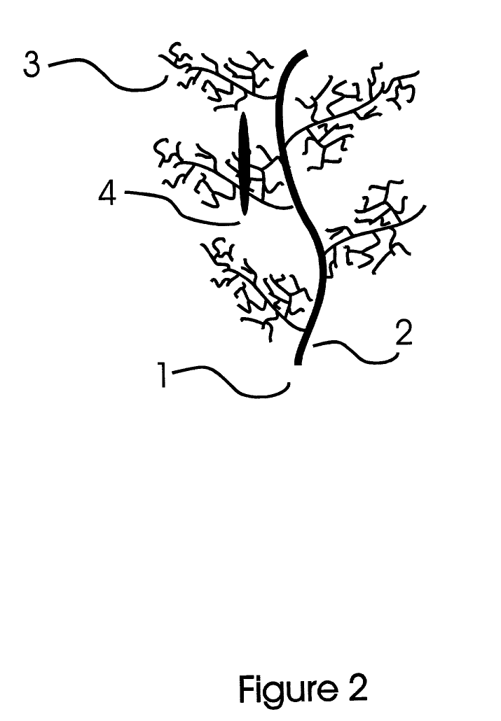

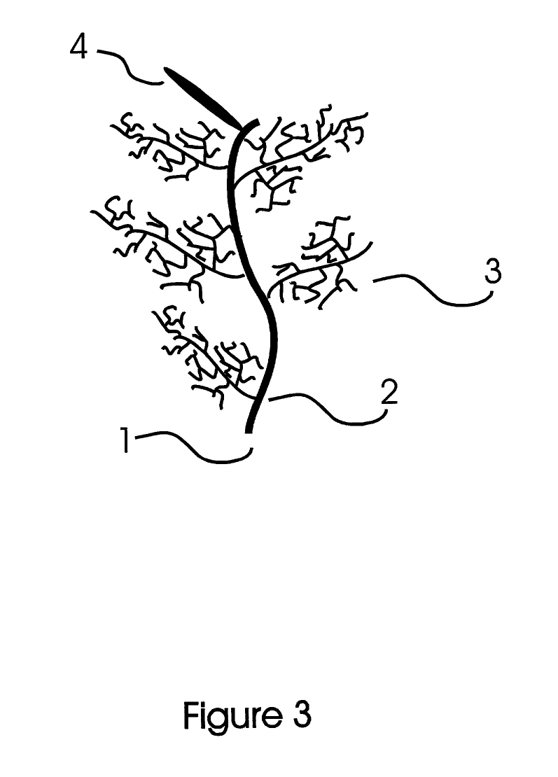

As shown in FIG. 2, the biologically active agent (4) can be bound to mucin (1) by being entangled in the side chains (3) or the protein backbone (2) of mucin (1). Alternatively, as shown in FIG. 3, the biologically active agent (4) can be bound covalently to mucin (1). While FIG. 3 shown the biologically active agent (4) bound to the protein backbone (2) of mucin, the biologically active agent (4) can also be bound to the carbohydrate side chains (3) of mucin (1). Furthermore, mucin (1) and the biologically active agent (4) can be joined by any means including, but not limited, to those selected from the group comprised of physical bonds, chemical bonds, coordinate bonds, ionic bonds, covalent bonds, disulfide bonds, amide linkages, physical entanglement, attachment through said additional molecules, and combinations thereof.

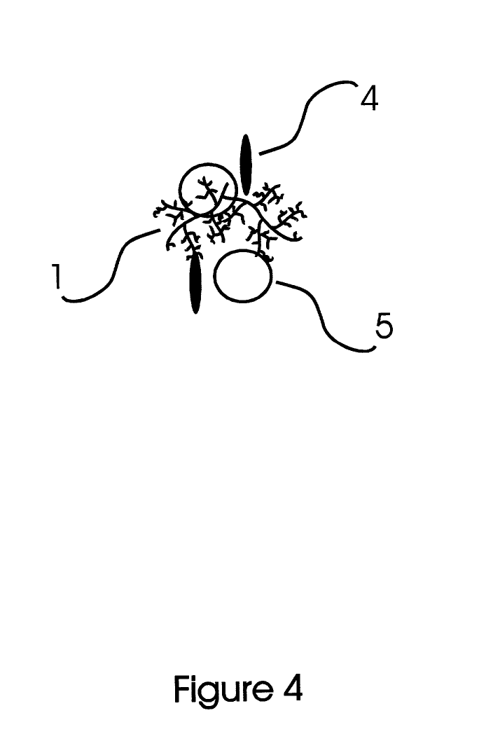

As shown in FIG. 4, the delivery vehicle for biologically active agents described in the present invention can optionally contain additional molecules (5) in addition to mucin (1) and the biologically active agent (4). Such additional molecules can serve any function from enhancing the activity of mucin (1) and/or the biologically active agent (4) to performing independent functions such as mediating controlled or targeted delivery of the delivery vehicle, according to the present invention. Mucin (1), the biologically active agent (4) and the additional molecules (5) can be joined together by a means selected from the group comprised of physical bonds, chemical bonds, ionic bonds, covalent bonds, coordinate bonds, disulfide bonds, amide linkages, physical entanglement, and combinations thereof. The additional molecules can be any types of molecules including, but not limited to, molecules selected from the group comprised of deoxyribonucleic acid, ribonucleic acid, peptide nucleic acid, polynucleotides, nucleic acids, genes, antisense deoxyribonucleic acid, antisense ribonucleic acid, proteins, peptides, lectins, enzymes, lipids, hormones, carbohydrates, polysaccharides, mucopolysaccharides, lipoproteins, glycoproteins, glycolipids, collagen, elastin, cells, ribozyme, synthetic polymers, synthetic monomers, biological polymers, biological monomers, biomolecules, metals, elements, synthetically created molecules, naturally occurring molecules and combinations thereof.

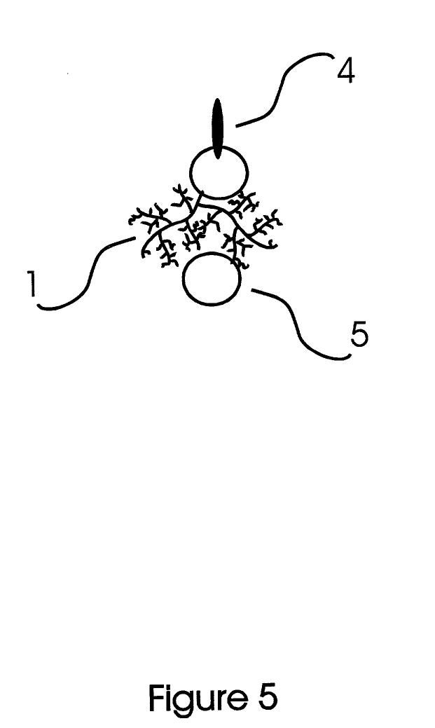

As shown in FIG. 5, mucin (1) and the biologically active agent (4) do not need to be in direct physical contact in the delivery vehicle, according to the present invention. In FIG. 5, mucin (1) and the biologically active agent (4) are both in contact with an additional molecule (5) but not with each other. While FIG. 5 shows one configuration of this concept, it is understood that many other such configurations can be developed for a biologically active agents delivery vehicle comprising mucin, according to the present invention. In the configuration shown in FIG. 5, mucin (1), while not in direct contact with the biologically active agent (4), still plays an important role in biomolecules delivery by enhancing the transport of the vehicle and providing specificity for cell recognition.

The mucin-comprising vehicle for the delivery of biologically active agents, as described in the present invention, can be used for any delivery applications for biologically active agents. Furthermore, the vehicle, can be modified by any means including, but not limited to, biological modification, physical modification, chemical modification, biochemical modification, enzymatic modification, heat-based modification, electrical current based modification, electrical charge based modification, pH based modification, temperature variation based modification, heat based modification, cold based modification, pressure based modification, and combinations thereof.

The delivery vehicle described in the present invention can be used to transport biologically active agents to specific cells, cell types, tissues, organs, organelles, or interstitial spaces in organisms. The targets for the delivery vehicle of the present invention can be cells selected from the group consisting of skin cells, brain cells, lung cells, liver cells, spleen cells, blood cells, mucus cells, muscle cells, heart cells, bone cells, bone marrow cells, thymus cells, heart cells, lymph cells, cartilage cells, pancreas cells, kidney cells, gall bladder cells, liver cells, stomach cells, intestine cells, testis cells, ovary cells, uterus cells, breast cells, rectum cells, nervous system cells, eye cells, gland cells, lymph node cells, connective tissue cells, skeletal system cells, nervous system cells, reproductive system cells, cardiovascular system cells, digestive system cells, immune system cells, urinary system cells, lymphatic system cells, and respiratory system cells.

The vehicle described in the present invention can be transported into cells, organisms or interstitial spaces in organisms or tissues using means including, but not limited to, means selected from the group consisting of delivering said biologically active agents subcutaneously, intradermally, intramuscularly, subdermally, intrathecally, transdermally, intravenously, orally, through inhalation, through insufflation, ocularly, rectally, vaginally, and into the interstitial spaces of tissues.

The mucin-containing delivery vehicle for the transport of biologically active agents, as described in the present invention, thus offers a new tool for the in vivo, ex vivo or in vitro, delivery of DNA, RNA, proteins and other biologically active agents into cells. The delivery vehicle for the transport of biologically active agents, as described in the present invention, can be used for many different applications including but not limited to applications selected from the group consisting of in vivo gene delivery, ex vivo gene delivery, in vitro gene delivery, gene therapy, vaccination, genetic vaccination, drug delivery, therapeutic agents delivery, biologically active agents delivery, pharmaceutical products delivery, protein delivery, peptide delivery, enzyme delivery, hormone delivery, cell repair, gene repair, DNA repair, cell modification, cell function restoration, gene expression, clinical testing, and diagnostic testing.

Furthermore, a delivery vehicle comprising mucin can also have other applications such as the development of binding assays and diagnostic kits or tests used to analyze the binding of specific biomolecules or chemical entities to other molecules. This technology can also be used in developing testing and laboratory kits for identifying the presence of certain biomolecules in organisms such as for urine, blood or other bodily secretion diagnostic assays for humans.

The mucin-based vehicle described in the present invention can be used for the transport of biologically active agents into organisms or cells of organisms where said organisms are selected from the group comprised of living eukaryotic organisms, non-living eukaryotic organisms, living prokaryotic organisms, non-living prokaryotic organisms, mammalian organisms, vertebrate organisms, invertebrate organisms, and microorganisms.

While a specific embodiment of the invention has been shown and described in detail to illustrate the application of the principles of the invention, it is understood that the invention may be embodied otherwise without departing from such principles and that various modifications, alternate constructions, and equivalents will occur to those skilled in the area given the benefit of this disclosure and the embodiment described herein, as defined by the appended claims.

What is claimed is:

1. A vehicle for the means of delivering biologically active agents into an organism wherein said vehicle comprises
a. mucin;
b. at least one biologically active agent; and,
wherein said vehicle optionally contains
c. additional molecules; and,
whereby said vehicle optionally delivers said biologically active agents to a specific target in said organism.

2. An organism of claim 1 wherein said organism is selected from the group comprised of living eukaryotic organisms, non-living eukaryotic organisms, living prokaryotic organisms, and non-living prokaryotic organisms.

3. An organism of claim 1 wherein said organism is selected from the group comprised of mammalian organisms, vertebrate organisms, invertebrate organisms, and microorganisms.

4. Mucin of claim 1 wherein said mucin is a mucin type selected from the group comprised of mucin monomers, mucin dimers of the same type of mucin, mucin trimers of the same type of mucin, mucin polymers of the same type of mucin, mucin dimers of different types of mucin, mucin trimers of different types of mucin, mucin polymers of different types of mucin, mucin copolymers, mucin homopolymers, linear mucin polymers, branched mucin polymers, cross-linked mucin polymers, mucin components, mucin subcomponents, mucin particles, mucin fragments, mucin protein backbone, mucin carbohydrate side chains, mucin conjugates, mucin conjugates with lipids, mucin conjugates with proteins, mucin conjugates with peptides, mucin conjugates with carbohydrates, and combinations thereof.

5. Mucin of claim 1 wherein said mucin is selected from the group comprised of native mucin; mucin from a biological source; mucin from a natural source; mucin from a living organism; mucin from a non-living organism; synthetically produced mucin; modified mucin; physically altered mucin; chemically altered mucin; biochemically altered mucin; biologically altered mucin; electrochemically altered mucin; enzyme modified mucin; purified mucin; mucin obtained from humans; mucin obtained from plants; mucin obtained from living organisms; mucin purified from humans; mucin purified from plants; mucin purified from living organisms; and, combinations thereof.

6. Mucin of claim 1 wherein said mucin is selected from the group comprised of biologically modified mucin; physically modified mucin; chemically modified mucin; enzyme modified mucin; mucin modified by variations in temperature; heat modified mucin; cold modified mucin; mucin modified by the alteration of electric charges on mucin; mucin modified by the alteration of electric charges on mucin protein components; mucin modified by the alteration of electric charges on mucin carbohydrate components; mucin modified by changes in pH; mucin modified by the addition of carbohydrate molecules to said mucin; mucin modified by the removal of carbohydrate molecules from said mucin; mucin modified by the addition of protein molecules to said mucin; mucin modified by the removal of protein molecules from said mucin; mucin modified by the addition of biomolecules to said mucin; mucin modified by the addition of lipid to said mucin; mucin modified by the application of pressure to said mucin; mucin modified by the application of an electric current to said mucin; and, combinations thereof.

7. Biologically active agents of claim 1 wherein said biologically active agents are one or more different biologically active agents selected from the group comprised of biologically active chemical compounds, deoxyribonucleic acid, ribonucleic acid, peptide nucleic acid, polynucleotides, nucleic acids, genes, antisense deoxyribonucleic acid, antisense ribonucleic acid, ribozymes, proteins, peptides, amino acids, lectins, enzymes, lipids, carbohydrates, polysaccharides, mucopolysaccharides, lipoproteins, glycoproteins, glycolipids, inhibitors, biological response modifiers, hormones, antibodies, monoclonal antibodies, antigens, biologically active chemical compounds, vitamins, cells, cell fragments, biotechnology drugs, blood products, cardiovascular agents, central nervous system agents, gastrointestinal agents, respiratory agents, ace-inhibitors, alkaloids, antacids, analgesics, anabolic agents, anti-cancer agents, anti-infective agents, anti-fungal agents, anti-viral agents, anti-bacterial agents, anti-protozoan agents, anti-parasitic agents, anti-tubercular agents, anti-inflammatory agents, antineoplastic agents, anti-allergic agents, histamines, anti-histamines, anti-coagulation agents, circulatory drugs, metabolic potentiators, anti-anginal agents, anti-diabetic agents, anti-rheumatic agents, narcotics, opiates, cardiac glycosides, neuromuscular blockers, sedatives, local anesthetics, general anesthetics, radioactive compounds, prodrugs, anti-arthritic agents, analgesic agents, anti-emetic agents, dermatological agents, immunosuppressive agents, ophthalmic agents, ocular agents, anti-arrhythmia agents, anti-asthmatic agents, anti-cholesterolemics, anticonvulsants, anticoagulants, antidepressants, anti-diarrheal preparations, anti-emetics, anti-hypertensives, antilipid agents, anti-manic agents, anti-migraine agents, anti-nauseants, anti-psychotic agents, anti-stroke agents, anti-thyroid preparations, anabolic drugs, anti-obesity agents, antipyretic agents, antispasmodic agents, anti-thrombotic agents, anti-tumor agents, anti-ulcer agents, anti-uremic agents, anxiolytic agents, appetite stimulants, appetite suppressants, beta-blocking agents, bronchodilators, cardiovascular agents, cerebral dilators, chelating agents, chemotherapeutic agents, cognition activators, contraceptives, coronary dilators, cough suppressants, decongestants, deodorants, dermatological agents, diabetes agents, diuretics, emollients, erythropoietic drugs, expectorants, fertility agents, growth regulators, hormone replacement agents, hyperglycemic agents, hypoglycemic agents, ion-exchange resins, laxatives, migraine treatments, mineral supplements, mucolytics, narcotics, neuroleptics, neuromuscular agents, non-steroidal anti-inflammatory agents, nutritional additives, peripheral vasodilators, prostaglandins, psychotropic agents, renin inhibitors, respiratory stimulants, sedatives, steroids, stimulants, sympatholytics, thyroid preparations, tranquilizers, uterine relaxants, vaginal preparations, vasoconstrictors, vasodilators, anti-vertigo agents, wound healing agents, and combinations thereof.

8. Biologically active agents of claim 1 wherein said biologically active agents are one or more types of biologically active agents selected from the group comprised of native biologically active agents, biological source derived biologically active agents, synthetically produced biologically active agents, modified biologically active agents, physically altered biologically active agents, chemically altered biologically active agents, electrochemically altered biologically active agents, enzyme modified biologically active agents, purified biologically active agents, native therapeutic agents, biological source derived therapeutic agents, synthetically produced therapeutic agents, modified therapeutic agents, physically altered therapeutic agents, chemically altered therapeutic agents, enzyme modified therapeutic agents, and purified therapeutic agents.

9. Mucin of claim 1 wherein said mucin undergoes modifications such that said vehicle delivers said biologically active agents to said specific targets.

10. Additional molecules of claim 1 wherein said additional molecules undergo modifications such that said vehicle delivers said biologically active agents to said specific targets.

11. The vehicle of claim 1 wherein said mucin and said biologically active agent are joined together by a means selected from the group comprised of physical bonds, chemical bonds, ionic bonds, covalent bonds, coordinate bonds, disulfide bonds, amide linkages, physical entanglement, attachment through said additional molecules, and combinations thereof.

12. The vehicle of claim 1 wherein said mucin, said biologically active agent and said additional molecules are joined together by a means selected from the group comprised of physical bonds, chemical bonds, ionic bonds, covalent bonds, coordinate bonds, disulfide bonds, amide linkages, physical entanglement, and combinations thereof.

13. The vehicle of claim 1 wherein said vehicle is modified using biological modification, physical modification, chemical modification, biochemical modification, enzymatic modification, heat-based modification, electrical current based modification, electrical charge based modification, pH based modification, temperature variation based modification, heat based modification, cold based modification, pressure based modification, and combinations thereof.

14. The vehicle of claim 1 wherein said vehicle delivers said biologically active agents into said organism for a means selected from the group comprised of enhancing delivery rate of said biologically active agent, regulating dosage of said biologically active agent, delivering said biologically active agent to specific cells, and regulating the rate of the delivery of said biologically active agent to the cell.

15. Additional molecules of claim 1 wherein said additional molecules are selected from the group comprised of deoxyribonucleic acid, ribonucleic acid, peptide nucleic acid, polynucleotides, nucleic acids, genes, antisense deoxyribonucleic acid, antisense ribonucleic acid, proteins, peptides, lectins, enzymes, lipids, hormones, carbohydrates, polysaccharides, mucopolysaccharides, lipoproteins, glycoproteins, glycolipids, collagen, elastin, cells, ribozyme, synthetic polymers, synthetic monomers, biological polymers, biological monomers, biomolecules, metals, elements, synthetically created molecules, naturally occurring molecules and combinations thereof.

16. The target of claim 1 wherein said target is selected from the group comprised of skin cells, brain cells, lung cells, liver cells, spleen cells, blood cells, mucus cells, muscle cells, bone cells, bone marrow cells, thymus cells, heart cells, lymph cells, cartilage cells, pancreas cells, kidney cells, gall bladder cells, liver cells, stomach cells, intestine cells, testis cells, ovary cells, uterus cells, breast cells, rectum cells, nervous system cells, eye cells, gland cells, lymph node cells, connective tissue cells, skeletal system cells, nervous system cells, reproductive system cells, cardiovascular system cells, heart cells, digestive system cells, immune system cells, urinary system cells, lymphatic system cells, and respiratory system cells.

17. The means for transporting the biologically active agent of claim 1 wherein said means is selected from the group consisting of delivering said biologically active agent subcutaneously, intradermally, intramuscularly, subdermally, intrathecally, transdermally, intravenously, orally, through inhalation, through insufflation, ocularly, rectally, vaginally, and into the interstitial spaces of tissues.

18. The vehicle of claim 1 wherein said vehicle is used for applications selected from the group consisting of in vivo gene delivery, ex vivo gene delivery, in vitro gene delivery, gene therapy, vaccination, genetic vaccination, drug delivery, therapeutic agents delivery, biologically active agents delivery, pharmaceutical products delivery, protein delivery, peptide delivery, enzyme delivery, hormone delivery, cell repair, gene repair, DNA repair, cell modification, cell function restoration, gene expression, clinical testing, and diagnostic testing.
